(19) United States
(12) Patent Application Publication
(10) Pub. No.: US 2023/0149915 A1
(43) Pub. Date: May 18, 2023

(54) CATALYST FOR OXIDATIVE COUPLING OF METHANE COMPRISING PALLADIUM SUPPORTED ON CERIUM PALLADIUM SOLID SOLUTION AND METHOD FOR OXIDATIVE COUPLING USING SAME

(71) Applicant: KOREA ADVANCED INSTITUTE OF SCIENCE AND TECHNOLOGY, Daejeon (KR)

(72) Inventors: Hyunjoo LEE, Seoul (KR); Gihun KWON, Daejeon (KR)

(21) Appl. No.: 16/970,682

(22) PCT Filed: May 29, 2020
(86) PCT No.: PCT/KR2020/007046

§ 371 Date: Aug. 18, 2020

(30) Foreign Application Priority Data
Sep. 30, 2019 (KR) 10-2019-0121114

(51) Int. Cl. B01J 37/00; C07C 2/84; B01J 23/63; B01J 6/00

(52) U.S. Cl. CPC B01J 37/00; C07C 2/84; B01J 23/63; B01J 6/001; C07C 2523/63

(57) ABSTRACT

Disclosed are: a catalyst for oxidative coupling of methane, the catalyst comprising palladium supported on a cerium palladium solid solution; and a method for oxidative coupling using the same, wherein highly oxidative Pd/CePdO and CePdO catalysts can be used in the production of C2 hydrocarbon compounds through oxidative coupling of methane, hereinafter OCM) at low temperatures.

### FIELD

The present disclosure relates to a catalyst for oxidative coupling of methane, the catalyst comprising palladium supported on a cerium palladium solid solution, and a method for oxidative coupling using the same and, more specifically, to a catalyst having palladium supported on a solid solution formed of cerium oxide and palladium oxide, a catalyst produced by subjecting the same to a leaching treatment, and a method for oxidative coupling of methane using the same.

The present disclosure was made with the support of the Ministry of Science and ICT in Korea under Project No. 2019000551, which was carried out in the program entitled “(EZBARO) Development of noble metal single atom-based heterogeneous catalyst for selective direct oxidation of methane (2019)” in the project named “Source technology development business” by the Korea Advanced Institute of Science and Technology under management of the National Research Foundation of Korea, from 1 Jan. 2019 to 31 Dec. 2019.

The present patent application claims priority to and the benefit of Korean Patent Application No. 10-2019-0121114 filed in the Korean Intellectual Property Office on 30 Sep. 2019, the disclosure of which is incorporated herein by reference.

### BACKGROUND

The concerns about the depletion of petroleum resources and the abundant reserves of shale gas are increasing interest in the selective conversion of methane gas as a main component thereof. Therefore, studies on the conversion of methane to another useful higher value-added compound (ethane, ethylene, methanol, or the like) are actively being conducted.

However, methane has a stable molecular structure (inertness) and strong C—H bonds, and thus existing methane conversion processes have the limitation of being an inefficient process since the activation of methane is attained in the process conditions of high temperatures (>1000 K) and high pressures (>30 bar) [Accounts Chem. Res. 2017, 50, 418-425].

Moreover, selective methane oxidation has many difficulties since a compound, such as methanol or ethane, as a product, is easily oxidized compared with methane [Nat. Mater. 2017, 16, 225-229].

Pd/CeO₂ catalyst in which Pd nanoparticles are dispersed on cerium oxide (ceria) has been widely used in the oxidation, such as Co oxidation, benzyl alcohol oxidation, and methane combustion. The Pd surface can be easily oxidized, and the formed PdO may act as an oxidation catalyst. The interface between Pd and cerium oxide often acts as an efficient active site for oxidation at low temperatures. It has been specifically reported that Pd can be highly oxidized on cerium oxide in which the ratio of Pd to O is smaller than 1. The methane activation of Pd has been studied, and it was found that the energy barrier was lower in PdO rather than the metal Pd.

A method for stably producing C2 compounds for a long time through oxidative coupling of methane (hereinafter, OCM) at low temperatures by using a highly oxidative Pd/CeO₂ catalyst has been provided, but the catalyst is easily reduced (inactivated) due to a limitation of oxygen activation in a catalytic reaction, resulting in a low rate of production of ethane.

### SUMMARY

### Technical Problem

The present inventors had made efforts for developing a method for stably producing C2 compounds for a long time by using methane at low temperatures. As a result, the present inventors had developed a method for producing C2 compounds through oxidative coupling of methane (hereinafter, OCM) at low temperatures by using highly oxidative Pd/CePdO and CePdO catalysts.

An aspect of the present disclosure is to provide a method for producing a catalyst for oxidative coupling of methane, the method including:

mixing a cerium oxide precursor solution and a palladium oxide precursor solution; and

subjecting a product of the mixing to calcination.

Another aspect of the present disclosure is to provide a catalyst for oxidative coupling of methane (OCM), the catalyst comprising palladium supported on CePdO solid solution.

Still another aspect of the present disclosure is to provide a method for oxidative coupling of methane, the method including adding to methane a catalyst for oxidative coupling of methane (OCM) to form a hydrocarbon compound comprising two or more carbon atoms from the methane, the catalyst comprising palladium supported on CePdO solid solution.

Still another aspect of the present disclosure is directed to use of a catalyst comprising palladium supported on CePdO solid solution for inducing oxidative coupling from methane.

### Technical Solution

The present disclosure relates to a catalyst for oxidative coupling of methane, the catalyst comprising palladium supported on a cerium palladium solid solution, and a method for oxidative coupling using the same.

As used herein, the term “solid solution” refers to a solid mixture having a continuously changing chemical composition within a predetermined range in the same crystal structure.

The present inventors had made efforts for developing a method for stably producing C2 compounds for a long time by using methane at low temperatures. As a result, the present inventors had developed a method for producing C2 compounds through oxidative coupling of methane (hereinafter, OCM) at low temperatures by using highly oxidative Pd/CePdO and CePdO catalysts.

As used herein, the term “Pd/CePdO catalyst” refers to a palladium catalyst supported on CePdO solid solution, and herein, the Pd/CePdO catalyst has the same meaning as Pd/CeₓPd₁₋ₓO_{2-y} catalyst.

As used herein, the term “CePdO solid solution” refers to a solid mixture having a continuously changing chemical composition of Ce, Pd, and O within a predetermined range in the same crystal structure, and herein, the CePdO solid solution has the same meaning as CeₓPd₁₋ₓO_{2-y} solid solution.

As used herein, the term “oxidative coupling” refers to a reaction in which two methane atoms are combined with each other to produce a hydrocarbon compound comprising C2, like in ethane or ethylene, or more carbon atoms.

As used herein, the term “hydrocarbon compound” refers to an organic compound composed of only carbon and hydrogen. The hydrocarbon compound includes aliphatic hydrocarbons (saturated hydrocarbons and unsaturated hydrocarbons), alicyclic hydrocarbons, and aromatic hydrocarbons.

As used herein, the term “C2 hydrocarbon compound” refers to a hydrocarbon compound having two carbon atoms. For example, the C2 hydrocarbon compound includes ethane, ethylene, acetylene, or the like, but is not limited thereto.

Hereinafter, the present disclosure will be described in detail.

In accordance with an aspect of the present disclosure, there is provided a method for producing a catalyst for oxidative coupling of methane (OCM), the method including:

mixing a cerium oxide precursor solution and a palladium oxide precursor solution; and

subjecting a product of the mixing to calcination.

As used herein, the term “calcination” refers to heating to high temperatures in air or oxygen. In the present disclosure, for oxidation of a catalyst at high temperatures through air, heat treatment, that is, calcination, was conducted.

The cerium oxide precursor solution may be an aqueous solution of (NH₄)₂Ce(NO₃)₆, Ce(NO₃)₃.6H₂O, CeCl₃, Ce(SO₄)₂, Ce(CH₃CO₂)₃, Ce(OH)₄, Ce₂(C₂O₄)₃ or a mixture of two or more thereof, and for example, (NH₄)₂Ce(NO₃)₆, but is not limited thereto.

The palladium oxide precursor solution is an aqueous solution of Pd(NO₃)₂, PdCl₂, or a mixture of two or more thereof.

The palladium oxide precursor solution may further contain nitro ethane (C₂H₅NO₂). The nitro ethane serves as a fuel in the calcination step at 350° C., thereby enabling the instant formation of a solid product together with flame.

The calcination step may be performed in the presence of air at 350-900° C., 600-900° C., or 600-800° C., and for example, 600-700° C., but is not limited thereto.

The calcination step may be performed for 12-48 hours, 12-36 hours, 12-24 hours, 12-18 hours, 16-48 hours, 16-36 hours, or 16-24 hours, and for example, 16-18 hours, but is not limited thereto.

In an embodiment of the present disclosure, when the calcination was conducted at a calcination temperature of 600-700° C. for 16-18 hours, PdO was sufficiently oxidized to show the highest reactivity.

The above method may further include a step of subjecting a product of the calcination step to a leaching treatment. For example, the leaching treatment may be conducted by immersing the product in nitric acid, but is not limited thereto.

As used herein, the term “leaching” refers to extracting a target component in a solid out of the solid through dissolution.

The leaching treatment step may be performed at 200-300° C., and for example, 225-275° C., but is not limited thereto.

The leaching treatment step may be performed for 1-3 hours, and for example, 1-2 hours, but is not limited thereto.

In an embodiment of the present disclosure, when the leaching treatment was performed at 225-275° C. for 1-2 hours, the Pd nanoparticles on the surface could be sufficiently leached.

In accordance with another aspect of the present disclosure, there is provided a catalyst for oxidative coupling of methane (OCM), the catalyst comprising palladium supported on CePdO solid solution.

According to an embodiment of the present disclosure, the catalyst for oxidative coupling of methane is PdO/CeₓPd₁₋ₓO_{2-y} in which x is 0<x<1 and y is 0≤y<2.

The palladium may be present as a particle on a surface of the catalyst, and may be present as an ion in the lattice of the catalyst.

In accordance with still another aspect of the present disclosure, there is provided a method for oxidative coupling of methane, the method including adding to methane a catalyst for oxidative coupling of methane (OCM) to form a hydrocarbon compound comprising two or more carbon atoms from the methane, the catalyst comprising palladium supported on CePdO solid solution.

According to the present disclosure, a hydrocarbon compound (for example, C2 hydrocarbon compounds, such as ethane) can be produced at low temperatures and a small amount of oxygen is used unlike a general methane oxidation reaction, leading to a significant reduction in costs in terms of a separation process.

According to an embodiment of the present disclosure, the hydrocarbon compound includes alkane, alkene, and alkyne compounds, wherein the alkane compound is a hydrocarbon compound of the molecular formula CₙH₂ₙ₊₂, the alkene compound is a hydrocarbon compound of the molecular formula CₙH₂ₙ, and the alkyne compound is a hydrocarbon compound of the molecular formula CₙH₂ₙ₋₂.

According to another embodiment of the present disclosure, the hydrocarbon compound is an alkane compound.

According to an embodiment of the present disclosure, the hydrocarbon compound is an alkane C2 hydrocarbon compound, and may be for example ethane, but is not limited thereto.

The step of forming a hydrocarbon compound may be performed by adding methane, oxygen, and the catalyst for oxidative coupling into a reactor.

According to an embodiment of the present disclosure, the reactor is selected from the group consisting of a fixed bed reactor, a fluidized bed reactor, and a membrane reactor.

The step of forming a hydrocarbon compound from methane of the present disclosure is performed in a temperature range of 390° C. or lower. According to an embodiment of the present disclosure, the step of forming a hydrocarbon compound of the present disclosure is performed in a temperature range of 230-390° C.

According to an embodiment of the present disclosure, the step of forming a hydrocarbon compound is performed by adding a moisture adsorbent.

According to an embodiment of the present disclosure, the moisture adsorbent is zeolite.

According to an embodiment of the present disclosure, the moisture adsorbent is a heat-treated moisture adsorbent.

According to an embodiment of the present disclosure, the heat-treated moisture adsorbent is a moisture adsorbent heat-treated at 100-500° C. More specifically, the heat-treated moisture adsorbent is a moisture adsorbent heat-treated at 100-200° C., 100-300° C., 100-400° C., 200-300° C., 200-400° C., 200-500° C., 300-400° C., 300-500° C., or 400-500° C. According to an example of the present disclosure, the step of forming a hydrocarbon compound by adding zeolite heat-treated at 300-400° C. is performed to improve the C₂H₆ production yield

According to an embodiment of the present disclosure, the catalyst for oxidative coupling is 5-100 mg in weight.

According to an embodiment of the present disclosure, the catalyst for oxidative coupling has a weight of 5-25 mg, 10-25 mg, 10-50 mg, or 25-100 mg.

According to an embodiment of the present disclosure, the step of forming a hydrocarbon compound is performed in a condition not including water.

According to an embodiment of the present disclosure, the step of forming a hydrocarbon compound is performed in a condition including 0.5-4% of water. More specifically, the step of forming a hydrocarbon compound is performed in a condition including 0.5-3%, 0.5-2%, 0.5-1%, 1-4%, 1-3%, 1-2%, 2-4%, or 2-3% of water. Most specifically, the step of forming a hydrocarbon compound is performed in a condition including 0.7-1.1% of water. According to an example of the present disclosure, the C₂H₆ production yield of the catalyst appeared to be high in a condition not including moisture.

### Advantageous Effects

According to the present disclosure, the present disclosure is directed to a catalyst for oxidative coupling of methane, the catalyst comprising palladium supported on a cerium palladium solid solution, and a method for oxidative coupling using the same, wherein highly oxidative Pd/CePdO and CePdO catalysts can be used in the production of C2 hydrocarbon compounds through oxidative coupling of methane, hereinafter OCM) at low temperatures.

### BRIEF DESCRIPTION OF THE DRAWINGS

FIG. 1 is a diagram showing a methane conversion reaction of a catalyst according to an example of the present disclosure.

FIG. 2 shows the diffuse reflectance infrared Fourier transform spectroscopy (CO-DRIFT) results regarding characteristics of Pd/CePdO and CePdO according to an example of the present disclosure.

FIG. 3A shows the transmission electron microscope (TEM) results regarding characteristics of Pd/CePdO according to an example of the present disclosure.

FIG. 3B shows the energy dispersive spectroscopy (EDS) mapping results regarding characteristics of Pd/CePdO according to an example of the present disclosure.

FIG. 3C shows the EDS mapping results regarding characteristics of Pd/CePdO according to an example of the present disclosure.

FIG. 3D shows the EDS mapping results regarding characteristics of Pd/CePdO according to an example of the present disclosure.

FIG. 3E shows the TEM results regarding characteristics of CePdO according to an example of the present disclosure.

FIG. 3F shows the EDS mapping results regarding characteristics of CePdO according to an example of the present disclosure.

FIG. 3G shows the EDS mapping results regarding characteristics of CePdO according to an example of the present disclosure.

FIG. 3H shows the EDS mapping results regarding characteristics of CePdO according to an example of the present disclosure.

FIG. 4A shows the X-ray photoelectron spectroscopy (XPS) results regarding characteristics of Pd/CePdO according to an example of the present disclosure.

FIG. 4B shows the XPS results regarding characteristics of CePdO according to an example of the present disclosure.

FIG. 5 shows the X-ray diffractometer (XRD) analysis results regarding characteristics of Pd/CePdO and CePdO according to an example of the present disclosure.

FIG. 6A shows the X-ray absorption near edge structure (XANES) analysis results regarding characteristics of Pd/CePdO and CePdO according to an example of the present disclosure.

FIG. 6B shows the extended X-ray absorption fine structure (EXAFS) spectrum analysis results regarding characteristics of Pd/CePdO and CePdO according to an example of the present disclosure.

FIG. 7 shows EXAFS spectrum analysis results regarding characteristics of Pd/CePdO according to an example of the present disclosure.

FIG. 8A shows the Ce 3d XPS analysis results regarding characteristics of Pd/CeO₂ according to an example of the present disclosure.

FIG. 8B shows the Ce 3d XPS analysis results regarding characteristics of Pd/CePdO according to an example of the present disclosure.

FIG. 8C shows the comparison of the Ce 3d XPS analysis results between Pd/CePdO and CePdO according to an example of the present disclosure.

FIG. 8D shows the O 1s XPS analysis results regarding characteristics of Pd/CeO₂ according to an example of the present disclosure.

FIG. 8E shows the O 1s XPS analysis results regarding characteristics of Pd/CePdO according to an example of the present disclosure.

FIG. 8F shows the comparison of the O 1s XPS results between Pd/CePdO and CePdO according to an example of the present disclosure.

FIG. 9 shows the XPS analysis results regarding post-reaction characteristics of Pd/CePdO catalyst according to an example of the present disclosure.

FIG. 10 shows the O₂-temperature programmed desorption (O₂-TPD) results regarding characteristics of CeO₂ and CePdO supports and Pd/CeO₂ and Pd/CePdO catalysts according to an example of the present disclosure.

FIG. 11A is a graph showing the reactivity comparison results regarding characteristics of Pd/CePdO and CeO₂ according to an example of the present disclosure.

FIG. 11B is a graph showing ethane selectivity comparison results regarding characteristics of Pd/CePdO and CeO₂ according to an example of the present disclosure.

FIG. 12 shows the H2-temperature programmed reduction (H₂-TPR) results regarding characteristics of Pd/CeO₂ and Pd/CePdO according to an example of the present disclosure.

FIG. 13A is a graph showing the reactivities of Pd/CePdO according to an example of the present disclosure by catalyst weights.

FIG. 13B is a graph showing the temperature differences of Pd/CePdO according to an example of the present disclosure by weights.

FIG. 14 is a graph showing the C₂H₆ yields of Pd/CePdO according to an example of the present disclosure by moisture contents.

FIG. 15 is a graph showing the C₂H₆ yields of Pd/CePdO according to an example of the present disclosure by the addition or non-addition of zeolite.

### DETAILED DESCRIPTION

Hereinafter, the present disclosure will be described in more detail with reference to the following examples. However, these examples are given for illustrating the present disclosure, and the scope of the present disclosure is not limited thereto.

Throughout the present specification, the “%” used to express the concentration of a specific material, unless otherwise particularly stated, refers to (wt/wt) % for solid/solid, (wt/vol) % for solid/liquid, and (vol/vol) % for liquid/liquid.

### Example 1: Synthesis of Catalysts

### 1-1. Synthesis of PdO/CeₓPd₁₋ₓO_{2-y} Catalyst

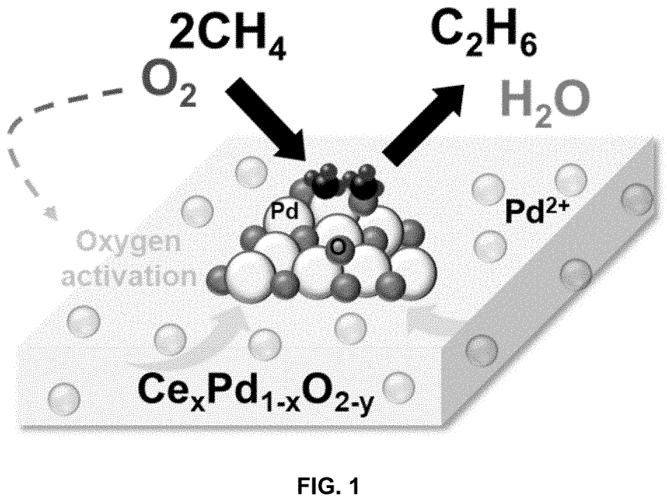

PdO/CeₓPd₁₋ₓO_{2-y} catalyst was typically synthesized by a solution-combustion method (FIG. 1). 1000 mg of (NH₄)₂Ce(NO₃)₆ (Sigma-Aldrich) was dissolved in 0.8 mL of deionized water to prepare a Ce-containing solution. 9 mg of Pd(NO₃)₂ and 340 mg of nitro ethane (C₂H₅NO₂) were added to 0.6 mL of deionized water, and the aqueous solution was dispersed in the Ce-containing solution to prepare a mixture.

The mixture was stirred to make a homogenous solution and moved to a crucible, and then the crucible was introduced into a furnace maintained at 350° C. Initially, the solution was boiled with frothing and foaming, and ignited to burn with a flame, yielding a solid product. The solid was ground in a mortar, and calcined at 650° C. in air for 16 hours. The sample was named “Pd/CePdO”.

### 1-2. Synthesis of CePdO Support

The calcined PdO/CeₓPd₁₋ₓO_{2-y} was subjected to a leaching treatment with nitric acid to prepare CeₓPd₁₋ₓO_{2-y} support.

Specifically, 0.1 g of PdO/CeₓPd₁₋ₓO_{2-y} was immersed in nitric acid (SAMCHUN, 60%) at 250° C. for 1 hour, and filtered with deionized water to remove residual NO₃⁻ in the sample. This leaching process was repeated three times to obtain clear CeₓPd₁₋ₓO_{2-y} support. The washed sample was dried at 80° C. overnight. Finally, CeₓPd₁₋ₓO_{2-y} support was successfully prepared without PdO particles on the surface thereof. The final CeₓPd₁₋ₓO_{2-y} sample was indicated as “CePdO”.

1-3. Synthesis of Pd/CeO₂ Catalyst

CeO₂ support as a comparative example was synthesized using a co-precipitation method. 1.0 g of Ce(NO₃)₃.6H₂O (99.99%, Kanto chemical) was dissolved in 23.5 mL of deionized water with slow stirring. Ammonia water (25-30% NH₄OH, Ducksan) was added dropwise until the pH of the solution reached 8.5. The produced yellow slurry was filtered, and the obtained precipitate was dried, and calcined at 773 K in air for 5 hours.

Pd/CeO₂ was synthesized by using a deposition-precipitation method. 0.38 g of CeO₂ powder was dispersed in 5 mL of deionized water. H₂PdCl₄ solution was prepared such that the molar ratio of PdCl₂ (99%, Sigma-Aldrich) and HCl (35-37%, Samchun) in deionized water was 1:2. Na₂CO₃ solution was prepared by dissolving 0.53 g of Na₂CO₃ (99.999%, Sigma-Aldrich) in 10 mL of deionized water. H₂PdCl₄ solution (up to 1 mL) containing 0.016 g of Pd was dropped in CeO₂ solution under rigorous stirring to produce 4 wt % of Pd/CeO₂ catalyst. Na₂CO₃ solution was also added to adjust the pH of the solution to approximately 9.

The final solution was stirred for 2 hours, and then aged at room temperature for 2 hours without stirring. This solution was filtered, and dried in an oven at 353 K for 5 hours. The produced Pd/CeO₂ catalyst was calcined at 750° C. in air for 25 hours.

### Example 2: Characterizations of Pd/CePdO and CePdO Supports

2-1. Characterizations of Pd/CePdO and CePdO Support by CO-DRIFT

To remove Pd on the surface of existing Pd/CePdO catalyst, the catalyst was subjected to a leaching treatment by being immersed in nitric acid at 250° C. for 1 hour, thereby obtaining CePdO support. The presence or absence of Pd on the surface of the sample was observed through CO-DRIFT analysis, by which the surface of the sample could be confirmed.

Specifically, the in-situ diffuse reflectance infrared Fourier transform spectroscopy (DRIFTS; Nicolet iS50, Thermo Scientific) measurement was carried out with an MCT detector and a diffuse reflectance assembly chamber having a KBr window. The sample was pretreated at 100° C. for 1 hour under Ar gas flow, and cooled to room temperature, and then a background spectrum was obtained. For CO adsorption, 1% CO/Ar gas flowed over the sample for 10 minutes to saturate CO.

The spectra were observed during CO desorption by Ar flow with evacuation for 20 minutes at room temperature. Finally, adsorbed-CO spectra onto the sample were obtained. The oxidation state of Pd was investigated by X-ray photoelectron spectroscopy (XPS, K-Alpha, Thermo VG Scientific). Binding energies were calculated using the maximum intensity of the advantageous C 1s signal at 284.8 eV as a reference.

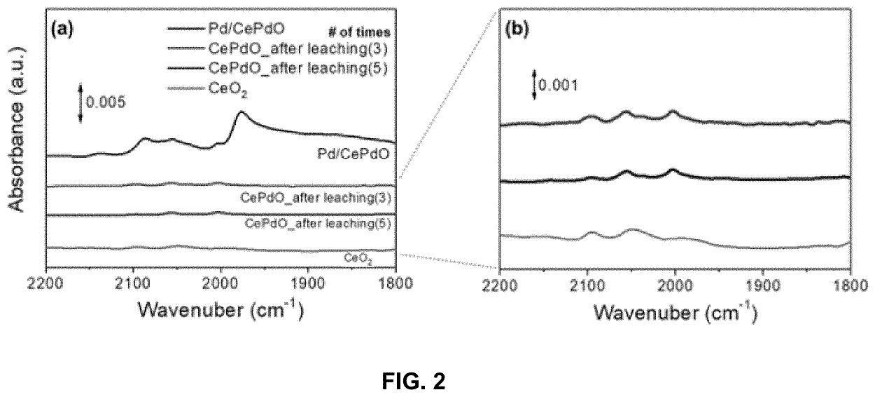

As can be confirmed in FIG. 2, the CO molecules adsorbed onto single Pd sites showed a peak at 2000-2200 cm⁻¹, while CO adsorbed onto ensemble Pd sites with a bridge mode or a 3-fold hollow mode showed a peak at 1900-2000 or 1800-1900 cm⁻¹, respectively.

The CO adsorption peak was observed in the Pd/CePdO catalyst, and thus in the Pd/CePdO catalyst, the CO molecules were adsorbed on single or ensemble Pd sites, showing the presence of Pd nanoparticles on the surface. While no peak for CO adsorption was observed in CePdO and CeO₂. The CO molecules hardly chemisorbed on the surface of CePdO and CeO₂, indicating that Pd nanoparticles were removed from CePdO.

2-2. Characterizations of Pd/CePdO and CePdO Support by TEM & EDS Mapping

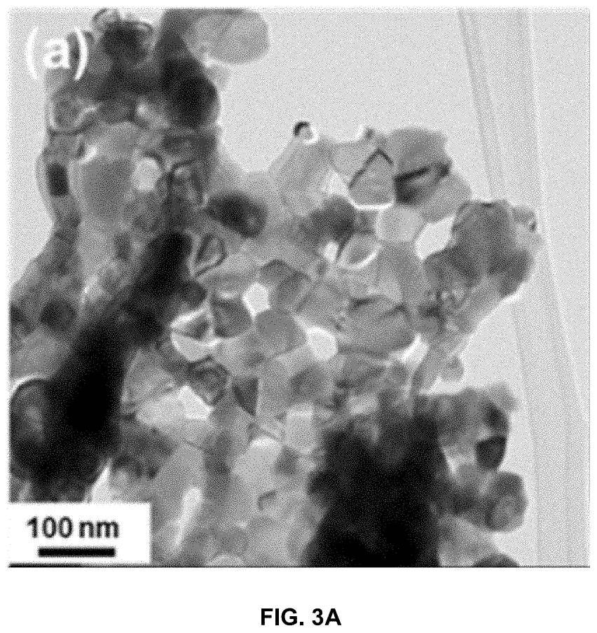

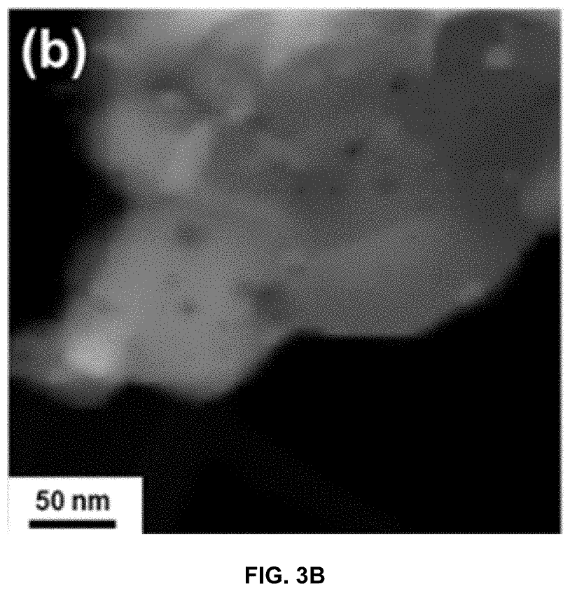

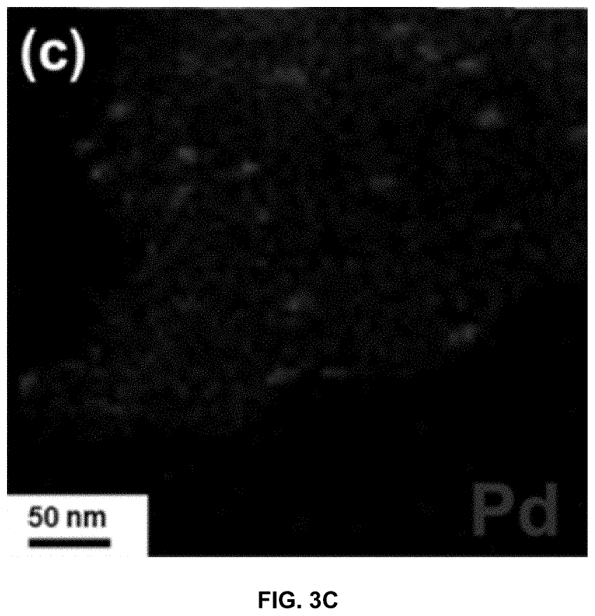

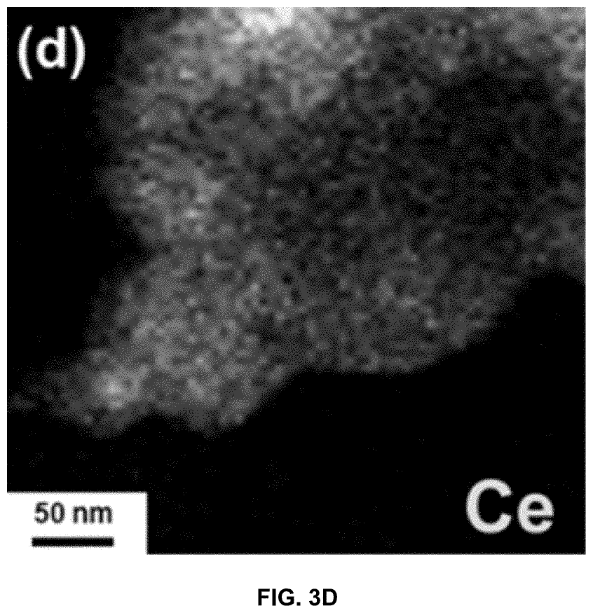

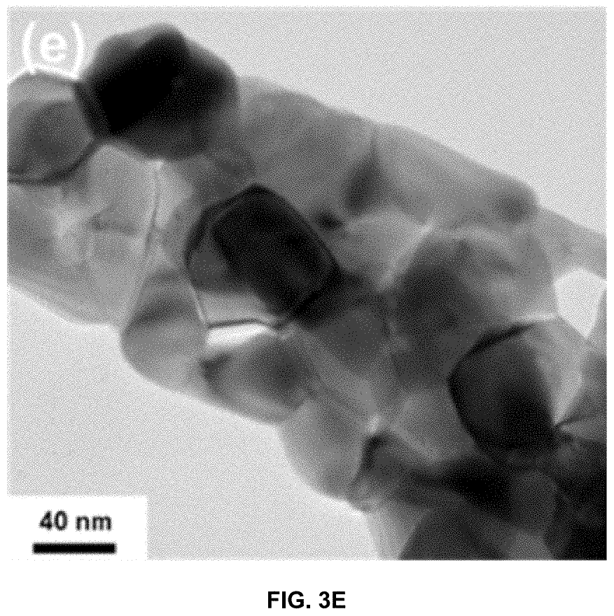

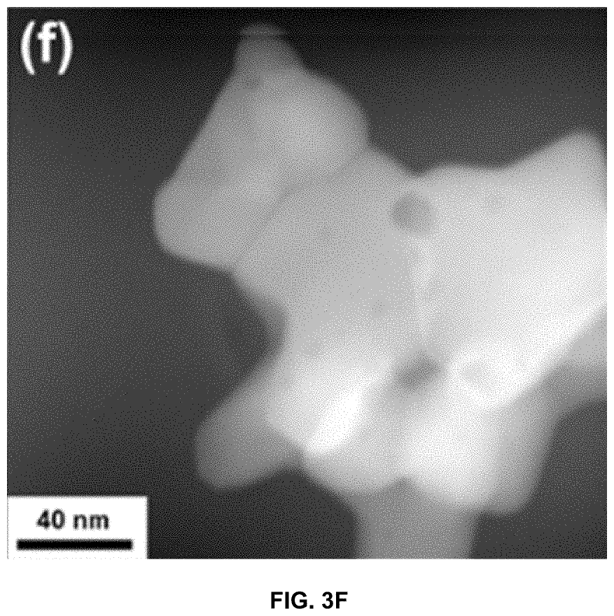

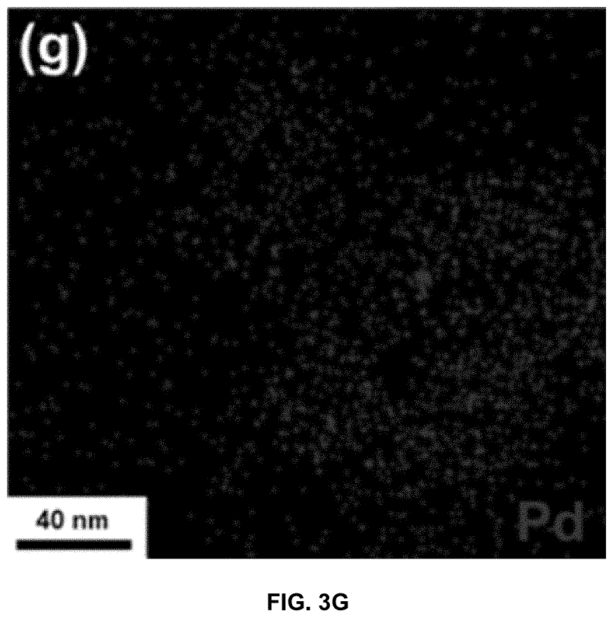

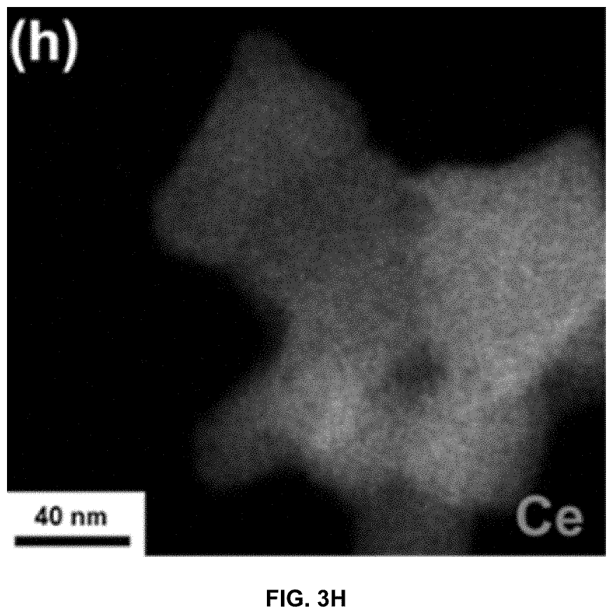

The Pd/CePdO catalyst and the CePdO support in section 2-1 above were analyzed by transmission electron microscope (TEM) (FIGS. 3A and 3E) & energy dispersive spectroscopy (EDS) (FIGS. 3B, 3C, 3D, 3F, 3G, and 3H).

High angle annular dark field-scanning TEM (HAADF-STEM) images and energy-dispersive X-ray spectroscopy (EDS) mapping images were obtained using a Titan cubed G2 60-300 (FEI) with an accelerating voltage of 200 kV (FIGS. 3A and 3E).

As can be confirmed in FIG. 3A, the catalyst was observed to have a size of about 50-70 nm.

As can be confirmed in FIGS. 3A to 3H, the density of Pd could be confirmed through EDS mapping analysis. As shown in FIG. 3C, clustered dots were observed in the existing Pd/CePdO catalyst, indicating the presence of Pd particles. Whereas, as shown in FIG. 3G, dots were not clustered but widely spread in CePdO. It could be indirectly guessed through these results that Pd particles were not present in CePdO but only Pd ions were spread out in the CePdO lattice.

2-3. Characterizations of Pd/CePdO and CePdO Support by XPS

The Pd/CePdO catalyst and the CePdO support in section 2-1 above were analyzed by X-ray photoelectron spectroscopy (XPS). The oxidation state of surface Pd could be confirmed through XPS analysis.

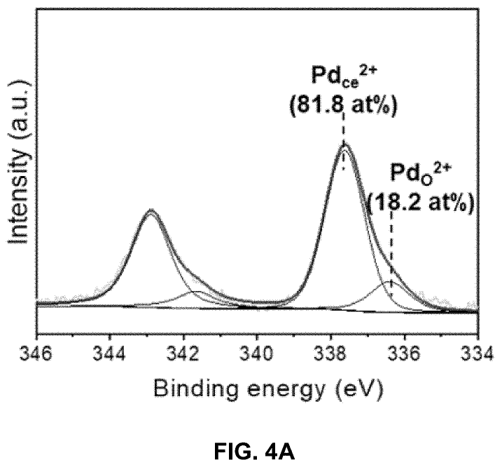

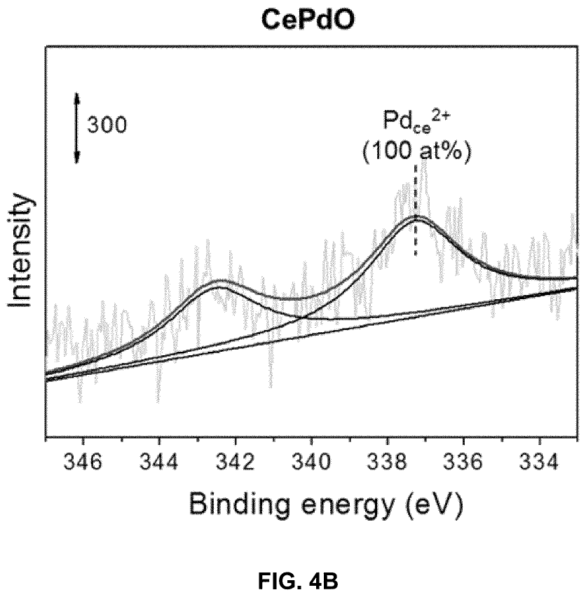

As can be confirmed in FIG. 4A, oxidized Pd²⁺ with strong intensity was mainly present in existing Pd/CePdO.

Whereas, as can be confirmed in FIG. 4B, only Pd_{Ce}²⁺(Pd—O—Ce) with very weak intensity was weakly observed in CePdO support subjected to a leaching treatment. It could be confirmed from these results that Pd ions were present in the CePdO lattice.

2-4. Characterizations of Pd/CePdO and CePdO Support by XRD

The Pd/CePdO catalyst and the CePdO support in section 2-1 above were analyzed by powder X-ray diffractometer (XRD, RIGAKU) in order to investigate crystalline structures before and after the leaching treatment.

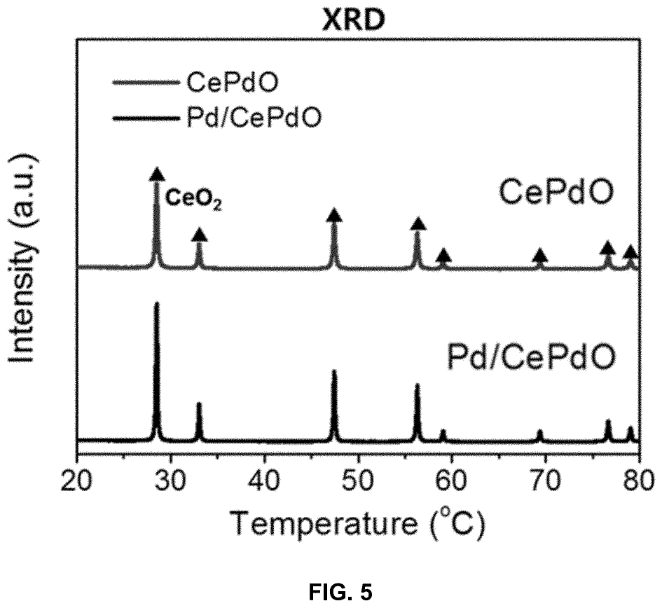

As can be confirmed in FIG. 5, only CeO₂ peak were mainly shown but neither PdO nor Pd peak was observed in the two samples (Pd/CePdO and CePdO). From the fact that no Pd peak was shown, it could be confirmed that separately clustered large Pd nanoparticles were not generated.

2-5. Characterizations of Pd/CePdO and CePdO Support by BET

The Pd/CePdO catalyst and the CePdO support in section 2-1 above were measured for BET surface area in order to investigate surface area change before and after the leaching treatment. Both of the Pd/CePdO and CePdO samples were concurrently analyzed to obtain results.

As can be confirmed in Table 1, both of the two samples similarly showed a low specific surface area of about 8 m²/g. It could be therefore seen that the change by a leaching treatment did not greatly affect the surface area.

TABLE 1

| — | Pd/CePdO | CePdO |
|---|---|---|
| BET surface area (m²/g) | up to 7.8 | up to 7.3 |

2-6. Characterizations of Pd/CePdO and CePdO Support by XANES and EXAFS

The Pd/CePdO catalyst and the CePdO support in section 2-1 above were analyzed by X-ray absorption near edge structure (XANES) and extended X-ray absorption fine structure (EXAFS) in order to investigate oxidation states and structures thereof.

The oxidation state of Pd could be confirmed by XANES analysis. XANES and EXAFS spectrum measurements were carried out at the 10C wide XAFS beam line of the Pohang Light Source (PLS). The energy of the storage ring electron beam was 2.5 GeV with a ring current of up to 360 mA. The incident X-ray was monochromatized by Si(111)/Si(311) double-crystals. The Pd K-edge spectra were obtained in a fluorescence mode using a passivate implanted planar silicon (PIPS) detector (Canberra). The spectrum for a reference Pd foil was also measured concurrently to calibrate each sample.

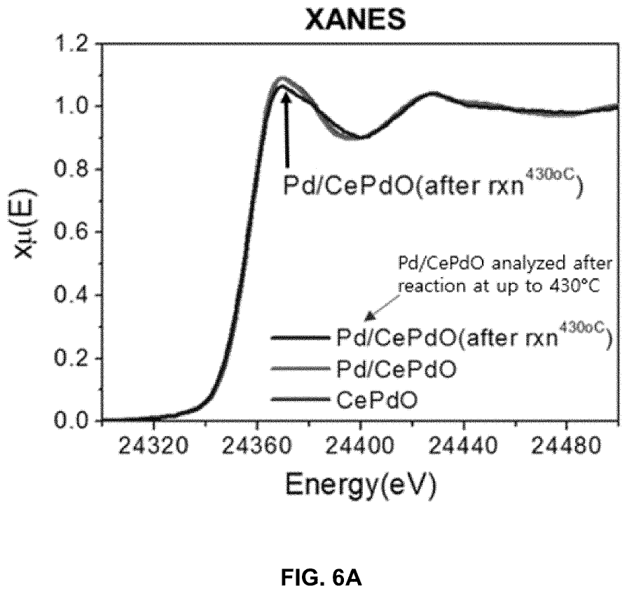

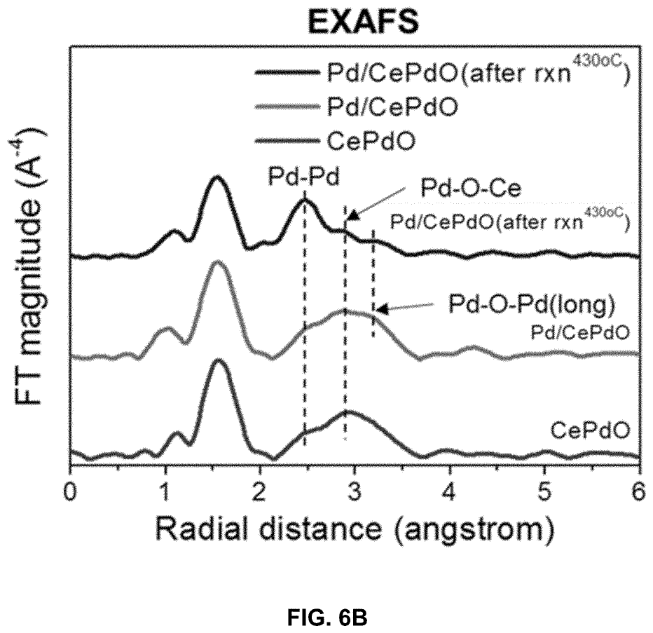

As can be confirmed from FIG. 6A, the oxidation states of Pd in Pd/CePdO and CePdO were similar, but in the Pd/CePdO catalyst performing a methane conversion reaction to 430° C., the reduction of Pd was confirmed through a reduction in the first peak (white line intensity) on the graph.

The structure of the sample can be confirmed by EXAFS analysis. The EXAFS and XANES data were processed and fitted with ARTEMIS and ATHENA softwares. A coordination number was calculated by fixing the S₀² to the values obtained from the reference Pd foil. The actual Pd amount in the catalysts was measured with an inductively coupled plasma optical emission spectrometer (ICP-OES, Agilent). The Pd content in the Pd/CePdO catalyst was about 1 wt %.

As can be confirmed from FIG. 6B, the peaks for Pd—O—Ce and Pd—O—Pd were mainly shown in Pd/CePdO, and only the peak for Pd—O—Ce was mainly shown in CePdO. Additionally, through the existence of the peak for Pd—Pd in Pd/CePdO after the methane conversion reaction to 430° C., the reduction of catalytic activity was confirmed to be due to the reduction of Pd.

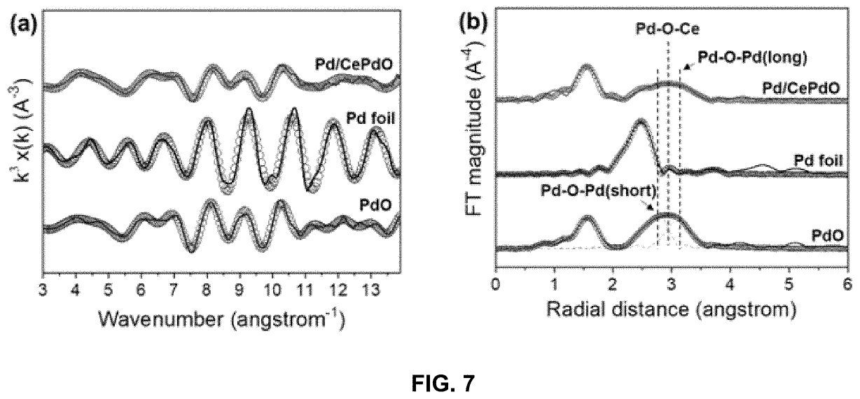

As can be seen from FIG. 7, it was re-confirmed that the peaks for Pd—O—Ce and Pd—O—Pd were mainly shown in Pd/CePdO. Table 2 shows the best fitting results to the EXAFS data. The coordination number for Pd—O—Ce was 3.2, indicating that Pd ions were introduced into the cerium oxide (ceria) lattice. The coordination number of Pd—O—Pd was 6.7 and these results indicate PdO nanoparticles formed on the surface of CePdO support.

TABLE 2

| | | Coordi- | | Debye- | |
|---|---|---|---|---|---|
| | Path | nation | Interatomic | Waller | R- |
| Sample | (Path) | number [R] | distance [A] | factor[δ²/A²] | factor |
| Pd/ | Pd—O | 4.4 | 2.006 | 0.003 | 0.029 |
| CePdO | Pd—Pd | 1.0 | 2.842 | 0.003 | |
| | Pd—O—Ce | 3.2 | 3.326 | 0.003 | |
| | Pd—O—Pd | 6.7 | 3.479 | 0.005 | |
| Pd | Pd—Pd | 12 | 2.742 | 0.006 | 0.004 |
| foil | Pd—Pd | 6 | 3.864 | 0.010 | |
| Bulk | Pd—O | 4.0 | 2.027 | 0.004 | 0.012 |
| PdO | Pd—O—Pd | 4.0 | 3.063 | 0.007 | |
| | Pd—O—Pd | 8.0 | 3.445 | 0.005 | |

### Example 3: Characteristic Comparison Between Pd/CePdO and Pd/CeO₂

3-1. Characteristic Comparison Between Pd/CePdO and Pd/CeO₂

X-ray photoelectron spectroscopy (XPS) for Ce 3d and O 1s was performed on Pd/CePdO and Pd/CeO₂. The change of oxygen vacancy sites were investigated through XPS analysis.

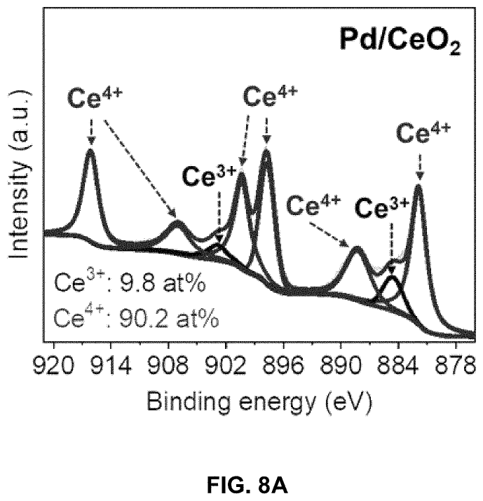

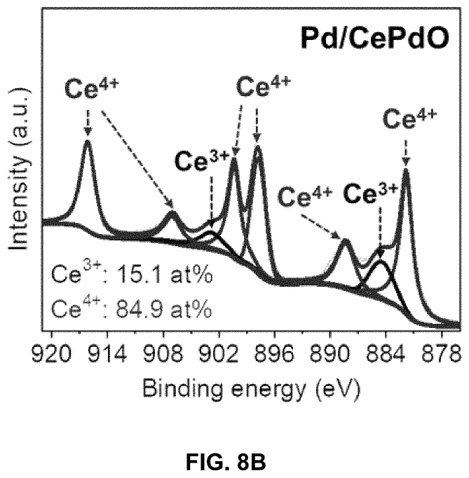

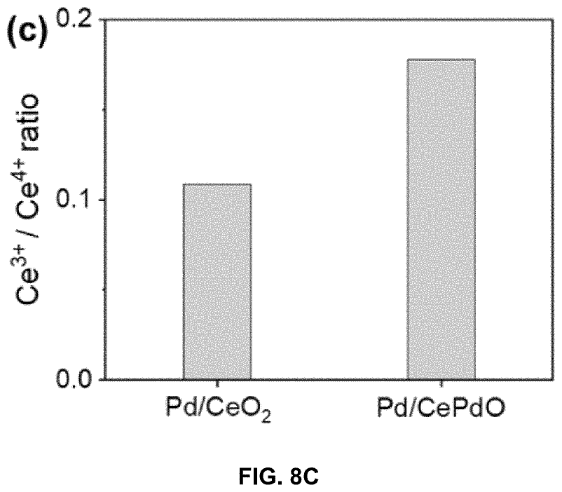

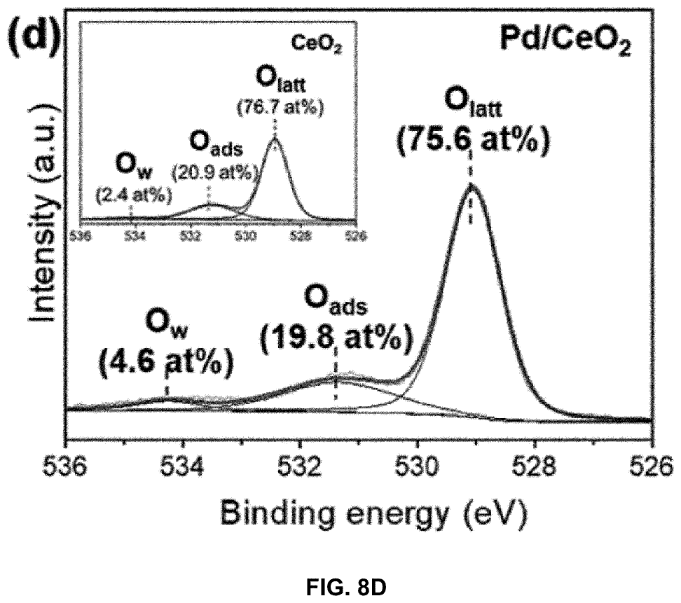

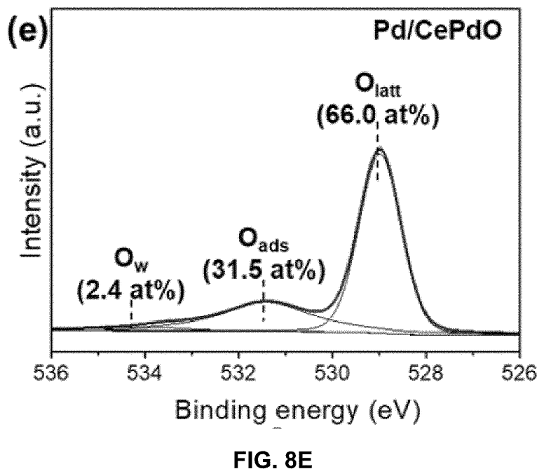

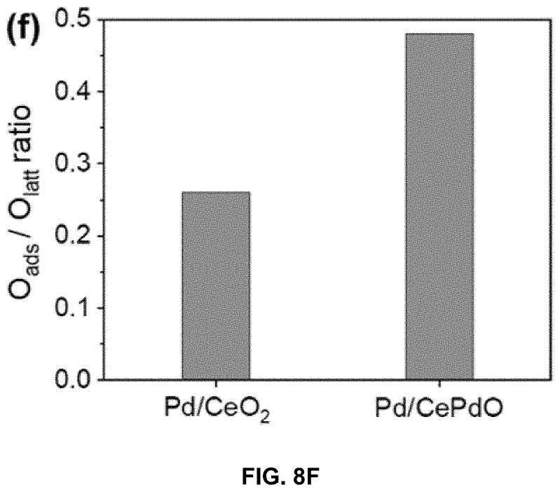

As can be confirmed from FIGS. 8A to 8C, the Ce 3d XPS peak was deconvoluted, and the percentages of Ce³⁺ and Ce⁴⁺ were estimated from the peak area ratio. The ratio of Ce³⁺ to Ce⁴⁺ was higher in Pd/CePdO (0.18) than in the Pd/CeO₂ (0.11). The higher Ce³⁺/Ce⁴⁺, the more oxygen vacancy sites were found, and the higher oxygen activation performance was obtained.

Similarly, as can be confirmed from FIGS. 8D to 8F, the O 1s XPS peak was deconvoluted to lattice oxygen (Oₗₐₜₜ), and the peak area ratio of the surface oxygen adsorbed on the defect sites (O_{ads}) was calculated. The O 1s peak was deconvoluted to Out at 529.0 eV; O_{ads} at 531.4 eV; and oxygen in the molecular water adsorbed on the catalyst surface (Ow) at 534.2 eV. When the ratio of O_{ads} to Oₗₐₜₜ was compared, Pd/CePdO showed a high value of 0.48 and the values for Pd/CeO₂ was 0.26, respectively. The above results indicate that the Pd/CePdO catalyst possesses abundant oxygen vacancy sites, promoting oxygen activation at lower temperatures.

3-2. Characteristic Comparison Between Pd/CePdO and Pd/CeO₂ by BET

To observe the change in surface area of Pd/CePdO and Pd/CeO₂, the BET surface area was measured.

As can be confirmed from Table 3, the surface Pd contents in Pd/CePdO and Pd/CeO₂ were 0.5 wt % and 4 wt %, respectively, and the Pd dispersions thereof were 29.6% and 38.1%, respectively. The BET surface areas thereof were 7.8 and 32.4 m²/g, respectively.

TABLE 3

| — | Pd/CePdO | Pd/CeO₂ |
|---|---|---|
| Pd content (wt %) | Up to 1^{a} | Up to 4^{c} |
| Pd dispersion (%) | 29.6^{b} | 38.1^{c} |
| BET surface area (m²/g) | Up to 7.8 | Up to 32.4 |

^{a}Surface Pd content in Pd/CePdO was 0.5 wt %, as measured by immersing in nitric acid at 250° C. The leaching solution with nitric acid was confirmed by ICP-OES analysis.
^{b}Pd dispersion was measured by pulsed CO chemisorption. Only surface Pd nanoparticles were considered. The CO-DRIFT results confirmed that CO was not adsorbed on CePdO and CeO₂.
^{c}ChemSusChem 2020 13 677-681.

### Example 4: Characterization of Pd/CePdO by XPS

Pd/CePdO was analyzed by X-ray photoelectron spectroscopy (XPS). The oxidation state of surface Pd could be confirmed through XPS analysis.

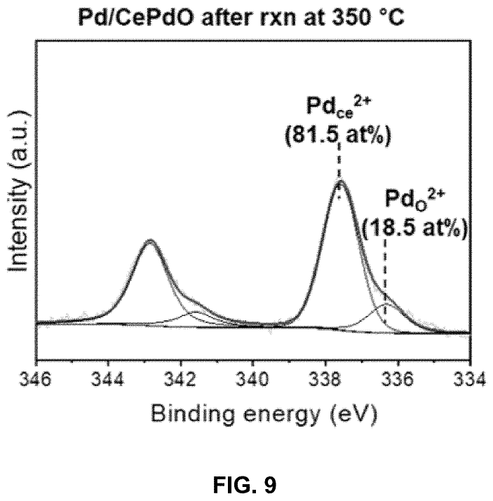

As can be confirmed from FIGS. 4B and 9, in XPS data, the oxidic Pd 3d_{5/2} peak was shown at 336.5 eV for Pd—O—Pd and 337.6 eV for Pd—O—Ce, while the metallic Pd 3d_{5/2} peak was shown at 335.5 eV. The Pd/CePdO catalyst showed dominant Pd_{Ce}²⁺ (Pd—O—Ce) sites as 81.8 at % from CePdO support, but Pd_{O}²⁺ (Pd—O—Pd) assigned to 18.2 at % by PdO nanoparticles as active sites for ethane production. The surface Pd state was maintained after the reaction at 350° C.

### Example 5: Investigation of Oxygen Transfer Ability of Pd/CePdO Catalyst

5-1. Investigation of Oxygen Transfer Ability of CeO₂ and CePdO Supports and Pd/CeO₂ and Pd/CePdO Catalysts

The oxygen transfer ability can be confirmed through O₂-TPD analysis, and the higher the peak at low temperatures, the better the oxygen transfer ability.

Specifically, the O₂-temperature programmed desorption (O₂-TPD) spectra were performed on a BETCAT-B (BEL, Japan) equipped with high-sensitivity TCD. The signals from water flowing through a water trap were excluded. The O₂-TPD spectra were obtained using 0.1 g of each catalyst. The catalysts were heated from room temperature to 900° C. with a ramping rate of 10° C. min⁻¹ under He gas flow.

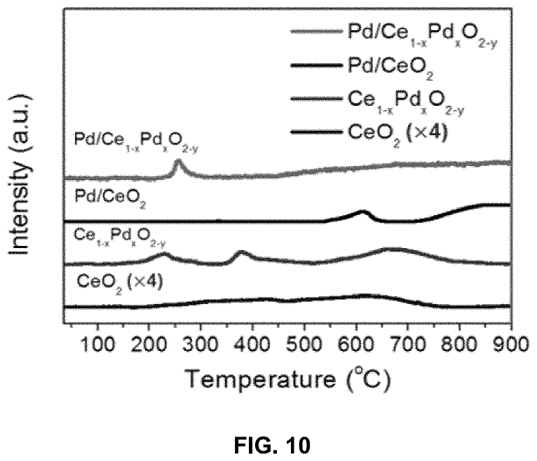

As can be confirmed from FIG. 10, the peak was shown at a low temperature in CePdO sample when CeO₂ and CePdO were compared. These results could confirm that CePdO had a higher oxygen transfer ability than existing CeO₂ (four-fold (×4) plot in order to distinguish resultant peaks of CeO₂ O₂-TPD).

In addition, when Pd/CePdO and Pd/CeO₂ were compared, the low peak was shown in the Pd/CePdO sample, indicating that Pd/CePdO had high oxygen transfer ability.

Additionally, as can be confirmed from FIG. 10, the bulk oxygen of Pd/CePdO was desorbed widely at 450° C. The above results also indicate that the oxygen transfer is more dominant than bulk oxygen desorption in Pd/CeO₂ at 720° C.

### Example 6: Reactivity Comparison Between Pd/CeO₂ and Pd/CePdO Catalysts

The reactivity of Pd/CePdO catalyst was compared with that of Pd/CeO₂ catalyst as a comparative example in actual methane reaction conditions.

Specifically, the reactivity of the catalysts was examined in a U-shaped quartz glass fixed-bed flow reactor at atmospheric pressure. The inlet gas was introduced with 6.8 scam of pure oxygen (99.995%, O₂), 8.4 scam of pure nitrogen (99.999%, N₂), and 90 sccm of pure methane (99.999% CH₄). N₂ gas was used as an internal standard. The amount of the catalyst used in the reaction was 10 mg. The reactor was heated at a ramping rate of 4° C. min⁻¹ and the temperature thereof was maintained for 2 hours, thereby establishing normal state conditions.

Since Pd/CePdO catalyst has lower surface factor (up to 7.8 m²g⁻¹) than Pd/CeO₂ (see the results of Example 3-2), the C₂H₆ productivity per surface Pd atom was estimated to conduct a comparison of the turnover frequency for ethane (TOF_{C2H6}).

The product gases (CO₂, C₂H₆, and a very small amount of C₂H₄) was analyzed by a gas chromatograph (GC-6100 series, Younglin) with Molecular Sieve 5A and Porapak N columns (Sigma-Aldrich) equipped with thermal conductivity detector (TCD) and flame ionization detector (FID) as a methanizer.

The Pd dispersion was measured through pulsed CO chemical adsorption, by using the modified Takeguchi's method. First, 25 mg of the Pd/CeO₂ catalyst was heated in the 5% O₂/He gas at 300° C. for 10 minutes, and then cooled to 50° C. while purging with He gas for 5 min. Thereafter, the catalyst was heated in 4.9% H₂/Ar gas to 200° C., and cooled to 50° C. Next, the catalyst was treated under following conditions; 1) He gas for 5 minutes, 2) 5% O₂/He gas for 5 minutes, 3) CO₂ gas for 10 minutes, 4) He gas for 20 minutes, 5) 4.9% H₂/Ar gas for 5 minutes. Finally, CO gas was pulsed every 1 minute in the He stream repeatedly until the adsorption of CO onto the catalyst was saturated.

C₂H₆ selectivity (%) was calculated by the following equation.

$${C}_{2} {H}_{6} selectivity ( \% ) = \frac{2 \cdot {C}_{2} {H}_{{6}_{out}}}{{[ {CO}_{2} + 2 \cdot {C}_{2} {H}_{6} ]}_{out}} \times 100 \qquad Equation 1$$

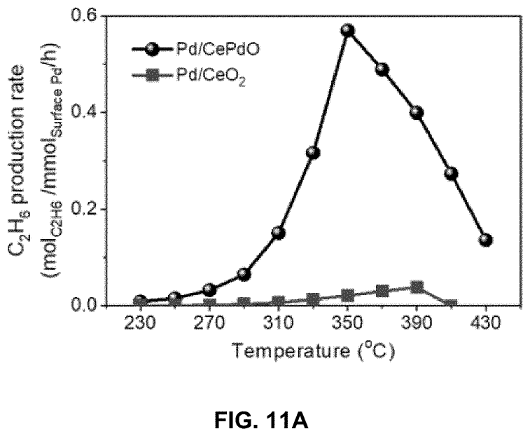

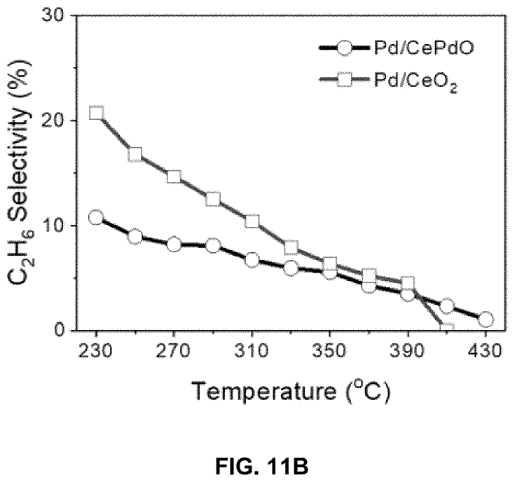

As can be confirmed from FIG. 11A and Table 4, as a result of reactivity test, the Pd/CePdO catalyst was not easily reduced (inactivated) even during the reaction and the reaction proceeded, showing a maximum ethane production rate (0.57 mol mmol_{surface Pd}⁻¹ h⁻¹) which was improved by about 27 times compared with the maximum rate of the Pd/CeO₂ catalyst at 350° C., and the ethane production rate was gradually decreased from 370° C.

The ethane production rate (TOF_{C2H6}) was calculated by the following equation.

$${C}_{2} {H}_{6} production rate = \frac{production {rate}_{C 2 H 6}}{{N}_{Pd} \times {D}_{Pd}} \times {m}_{Pd} \qquad Equation 2$$

Production rate_{C2H6} (mol/g_{cat}/h)
N_{pd}: Total Pd amount (mmol)
D_{pd}: Pd dispersion (%)
M_{pd}: Amount of catalyst used (g)

TABLE 4

| | Pd/CePdO | Pd/CeO₂ |
|---|---|---|
| Temperature | (mol mmol_{surface Pd}⁻¹ h⁻¹) | (mol mmol_{surface Pd}⁻¹ h⁻¹) |
| 230° C. | 0.01 | 0.00 |
| 250° C. | 0.02 | 0.00 |
| 270° C. | 0.03 | 0.00 |
| 290° C. | 0.06 | 0.00 |
| 310° C. | 0.15 | 0.01 |
| 330° C. | 0.32 | 0.01 |
| 350° C. | 0.57 | 0.02 |
| 370° C. | 0.49 | 0.03 |
| 390° C. | 0.40 | 0.04 |
| 410° C. | 0.27 | 0.00 |
| 430° C. | 0.14 | — |

As confirmed in FIGS. 6A and 6B with respect to Example 2-6 that Pd was reduced through the results in which reduced Pd (large Pd—Pd peak, FIG. 6A) was shown unlike the oxidized Pd in existing synthesized Pd/CePdO and CePdO by the measurement through XANES and EXAFS analysis after a methane conversion reaction to 430° C., the ethane production rate was reduced from the methane conversion reaction at 370° C.

As can be confirmed from FIG. 11B and Table 5, an excess of CO₂ was produced, resulting in relatively low ethane selectivity. Therefore, the possibility that a high ethane production rate can be sufficiently attained by favorably controlling necessary catalyst characteristics (O₂ activation) was confirmed in the present disclosure.

TABLE 5

| Temperature | Pd/CePdO (%) | Pd/CeO₂ (%) |
|---|---|---|
| 230° C. | 11 | 21 |
| 250° C. | 8.9 | 17 |
| 270° C. | 8.2 | 15 |
| 290° C. | 8.1 | 13 |
| 310° C. | 6.7 | 10 |
| 330° C. | 5.9 | 7.9 |
| 350° C. | 5.6 | 6.4 |
| 370° C. | 4.3 | 5.2 |
| 390° C. | 3.5 | 4.5 |
| 410° C. | 2.3 | 0 |
| 430° C. | 1 | — |

### Example 7: Characteristic Comparison Between Pd/CeO₂ and Pd/CePdO Catalyst by H₂-TPR

The H₂-temperature programmed reduction (H₂-TPR) spectra were obtained using BEL-CAT-II (BEL Japan Inc.) equipped with a thermal conductivity detector (TCD). 50 mg of each catalyst was heated for 1 hour at 200° C. in an Ar gas flow and cooled down to −90° C. using a cryogenic apparatus using liquid nitrogen. Then, the catalysts were exposed to a 5% H₂/Ar gas flow and stabilized under the flow for 30 minutes. The temperature increased from −90° C. to 900° C. at a ramping rate of 10° C. min⁻¹.

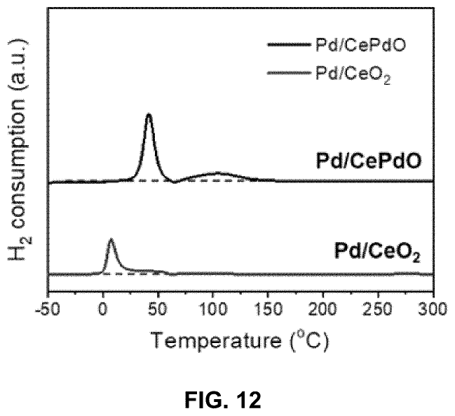

As can be confirmed from FIG. 12, Pd/CeO₂ was reduced at 7° C., and Pd nanoparticles supported on CePdO were reduced at 40° C.

The Pd/CePdO can maintain the oxidic Pd state in a reductive condition, and the CePdO support allows the Pd domain to maintain the oxidized state.

Since the oxidized Pd site was the active site for C₂H₆ production, the Pd/CePdO catalyst showed excellent TOF_{C2H6}.

The H₂-TPR results indicate that the C₂H₆ productivity gradually decreased in Pd/CePdO when the temperature was 350° C. or higher (see FIG. 11) was due to high tolerance to the reduction of PdO in a high concentration of CH₄.

### Example 8: Optimization of Catalyst Reaction Conditions for Maximizing C₂H₆ Yield

8-1. Catalyst Weight and Moisture Condition Affecting C₂H₆ Yield

For maximization of C₂H₆ yield, the reaction was performed by adjusting the weight of the catalyst to 5, 10, 25, 50, and 100 mg. The amounts of gases used in the reaction were the same as the amounts used in Example 6.

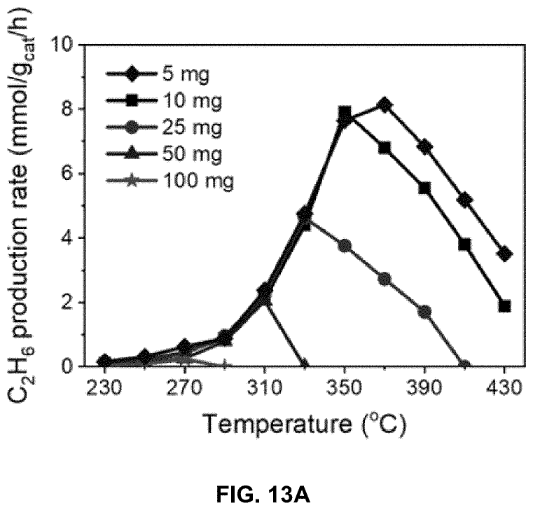

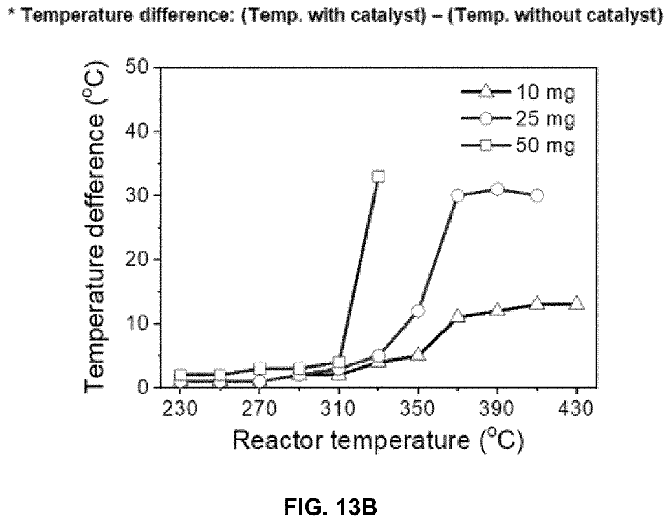

As can be confirmed in FIG. 13A and Table 6, the C₂H₆ production rate tended to decrease as the catalyst weight increased, and the reasons is that the catalyst was easily decomposed by temperature as the weight of the catalyst increased.

The ethane production rate was calculated by the following equation.

$${C}_{2} {H}_{6} production rate ( mmol / {g}_{cal} / h ) = \frac{Ethane production \times P \times 60 ( min / h )}{R \times T \times {m}_{Pd}} \qquad Equation 3$$

Ethane production (scam; mL/min)

P: Pressure (atm)

R: Gas constant (atm·L/mol/K)

T: Temperature (K)

M_{pd}: Amount of catalyst used (g)

TABLE 6

| | Catalyst | Catalyst | Catalyst | Catalyst | Catalyst |
|---|---|---|---|---|---|
| | weight | weight | weight | weight | weight |
| Temperature | 5 mg | 10 mg | 25 mg | 50 mg | 100 mg |
| 230° C. | 0.16 | 0.12 | 0.10 | 0.06 | 0.05 |
| 250° C. | 0.31 | 0.22 | 0.19 | 0.12 | 0.09 |
| 270° C. | 0.63 | 0.45 | 0.41 | 0.26 | 0.24 |
| 290° C. | 0.89 | 0.90 | 0.97 | 0.79 | 0.00 |
| 310° C. | 2.38 | 2.10 | 2.23 | 2.05 | — |
| 330° C. | 4.75 | 4.40 | 4.63 | 0.00 | — |
| 350° C. | 7.65 | 7.92 | 3.76 | — | — |
| 370° C. | 8.14 | 6.80 | 2.73 | — | — |
| 390° C. | 6.84 | 5.56 | 1.71 | — | — |
| 410° C. | 5.19 | 3.80 | 0.00 | — | — |
| 430° C. | 3.51 | 1.89 | — | — | — |

In addition, the weight of the catalyst and the thermal gradient (temperature difference) by the degradation of the catalyst were investigated through tests. The amounts of gases used in the reaction were the same as those used in Example 6, and the amounts of the catalyst used were 10, 25, and 50 mg.

As can be confirmed from FIG. 13B and Table 7, a high thermal gradient was shown as the weight of the catalyst increased, and thus the catalyst was easily degraded at low temperatures.

TABLE 7

| | Catalyst weight | Catalyst weight | Catalyst weight |
|---|---|---|---|
| Temperature | 10 mg | 25 mg | 50 mg |
| 230° C. | 1 | 1 | 2 |
| 250° C. | 1 | 1 | 2 |
| 270° C. | 1 | 1 | 3 |
| 290° C. | 2 | 2 | 3 |
| 310° C. | 2 | 3 | 4 |
| 330° C. | 4 | 5 | 33 |
| 350° C. | 5 | 12 | — |
| 370° C. | 11 | 30 | — |
| 390° C. | 12 | 31 | — |
| 410° C. | 13 | 30 | — |
| 430° C. | 13 | — | — |

To investigate the effect of moisture on catalyst activity, the reaction was performed by adjusting the amount of moisture to 0, 0.9, and 3.6 vol % in conditions containing 100 mg of a catalyst and 10 g of silica sand.

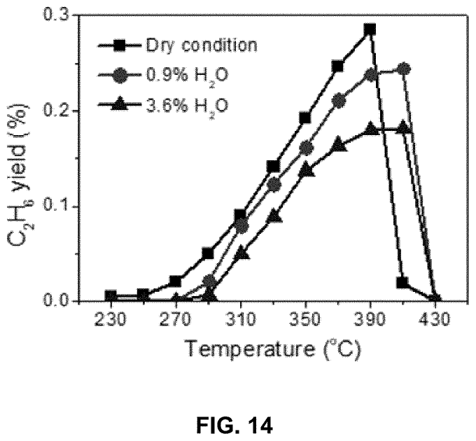

As can be confirmed from FIG. 14 and Table 8, the C₂H₆ yield was reduced as the amount of moisture increased.

The ethane production rate was calculated by the following equation.

$${C}_{2} {H}_{6} yield = \frac{2 \cdot {C}_{2} {H}_{{6}_{out}}}{{[ {CH}_{4} ]}_{ln}} \times 100 \qquad Equation 4$$

TABLE 8

| Temperature | Drying condition (%) | 0.9% H₂0 (%) | 3.6% H₂0 (%) |
|---|---|---|---|
| 230° C. | 0.00 | 0 | 0 |
| 250° C. | 0.01 | 0 | 0 |
| 270° C. | 0.02 | 0.00 | 0.00 |
| 290° C. | 0.05 | 0.02 | 0.01 |
| 310° C. | 0.09 | 0.08 | 0.05 |
| 330° C. | 0.14 | 0.12 | 0.09 |
| 350° C. | 0.19 | 0.16 | 0.14 |
| 370° C. | 0.25 | 0.21 | 0.16 |
| 390° C. | 0.28 | 0.24 | 0.18 |
| 410° C. | 0.02 | 0.24 | 0.18 |
| 430° C. | 0.00 | 0.00 | 0.00 |

8-2. Addition of Zeolite 13X for Maximizing C₂H₆ Yield

The thermal gradient (temperature difference) due to an excess of catalyst is known to be overcome when silica sand is added to the catalyst. Zeolite 13X (ThermoFisher Scientific, Massachusetts, USA) was used as a substance which takes the place of silica sand helpful in the reaction of the catalyst and adsorbs moisture.

The reaction conditions were controlled to 100 mg of catalyst and 53 sccm of total feed flow (with 73% CH₄ and 18% O₂). The Pd/CePdO catalyst and zeolite 13X were mixed, followed by reaction, and the pretreatment was performed at 200° C., 300° C., and 400° C. before the reaction was performed.

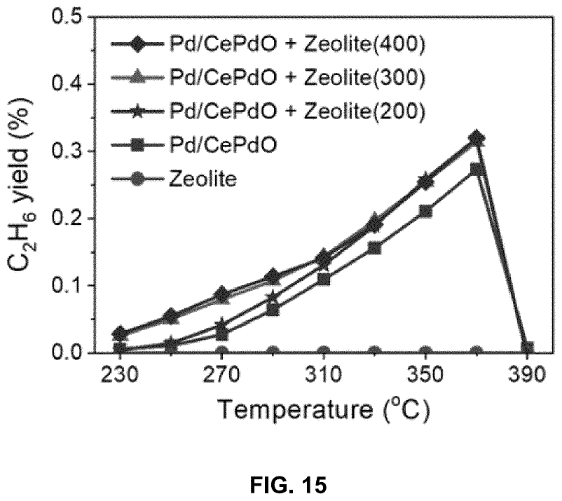

As can be confirmed from FIG. 15 and Table 9, the C₂H₆ yield was shown to increase when Pd/CePdO catalyst and zeolite 13X were mixed and reacted rather than when Pd/CePdO catalyst was used alone. In addition, the C₂H₆ yield increased by at least three times when zeolite 13X was added to perform a reaction rather than when silica sand was added.

TABLE 9

| | Pd/CePdO + | Pd/CePdO + | Pd/CePdO + | Pd/ | |
|---|---|---|---|---|---|
| Temper- | Zeolite(400) | Zeolite(300) | Zeolite(200) | CePdO | Zeolite |
| ature | (%) | (%) | (%) | (%) | (%) |
| 230° C. | 0.028 | 0.025 | 0.004 | 0.006 | 0.000 |
| 250° C. | 0.055 | 0.051 | 0.014 | 0.011 | 0.000 |
| 270° C. | 0.087 | 0.080 | 0.042 | 0.027 | 0.000 |
| 290° C. | 0.114 | 0.107 | 0.083 | 0.064 | 0.000 |
| 310° C. | 0.142 | 0.144 | 0.131 | 0.109 | 0.000 |
| 330° C. | 0.191 | 0.197 | 0.189 | 0.156 | 0.000 |
| 350° C. | 0.254 | 0.255 | 0.259 | 0.211 | 0.000 |
| 370° C. | 0.320 | 0.314 | 0.319 | 0.273 | 0.000 |
| 390° C. | 0.000 | 0.000 | 0.000 | 0.000 | 0.000 |
| 410° C. | — | — | — | — | — |
| 430° C. | — | — | — | — | — |

Consequently, the Pd/Ce₁₋ₓPdₓO_{2-y} catalyst can be used for the purpose of ethane direct conversion from methane by promoting oxygen activation or transfer. Furthermore, the production of ethane using Pd/Ce₁₋ₓPdₓO_{2-y} catalyst can be performed in conditions of lower than 400° C. and atmospheric pressure. Furthermore, the yield of ethane was increased when Pd/Ce₁₋ₓPdₓO_{2-y} catalyst was used together with a moisture adsorbent (zeolite 13X).

What is claimed is:

1. A method for producing a catalyst for oxidative coupling of methane (OCM), the method comprising:
mixing a cerium oxide precursor solution and a palladium oxide precursor solution; and
subjecting a product of the mixing to calcination.

2. The method of claim 1, wherein the cerium oxide precursor solution is an aqueous solution of (NH₄)₂Ce(NO₃)₆, Ce(NO₃)₃.6H₂O, CeCl₃, Ce(SO₄)₂, Ce(CH₃CO₂)₃, Ce(OH)₄, Ce₂(C₂O₄)₃ or a mixture of two or more thereof.

3. The method of claim 1, wherein the palladium oxide precursor solution is an aqueous solution of Pd(NO₃)₂, PdCl₂, or a mixture of two or more thereof.

4. The method of claim 3, wherein the palladium oxide precursor solution further contains nitro ethane (C₂H₅NO₂).

5. The method of claim 1, wherein the calcination is performed in the presence of air at 350-900° C.

6. The method of claim 1, wherein the calcination is performed for 48 hours or less.

7. A catalyst for oxidative coupling of methane (OCM), the catalyst comprising palladium supported on CePdO solid solution.

8. The catalyst of claim 7, wherein the catalyst for oxidative coupling of methane is PdO/CeₓPd₁₋ₓO_{2-y} in which x is 0<x<1 and y is 0≤y<2.

9. The catalyst of claim 7, wherein the palladium is present as a particle on a surface of the catalyst.

10. The catalyst of claim 7, wherein the palladium is present as an ion in the lattice of the catalyst.

11. A method for oxidative coupling of methane, the method comprising adding to methane a catalyst for oxidative coupling of methane (OCM) to form a hydrocarbon compound comprising two or more carbon atoms from the methane, the catalyst comprising palladium supported on CePdO solid solution.

12. The method of claim 11, wherein the oxidative coupling is performed by adding methane, oxygen, and the catalyst for oxidative coupling in a reactor.

13. The method of claim 12, wherein the oxidative coupling is performed at 230-390° C.

14. The method of claim 11, wherein the oxidative coupling is performed by adding a moisture adsorbent.

15. The method of claim 11, wherein the catalyst for oxidative coupling has a weight of 5-100 mg.

16. The method of claim 11, wherein the oxidative coupling is performed in a condition not including water.
